(19) United States
(12) Patent Application Publication
(10) Pub. No.: US 2024/0293305 A1
(43) Pub. Date: Sep. 5, 2024

(54) COMPOSITION WITH ANTIMICROBIAL OR ANTIFUNGAL ACTIVITY COMPRISING FERMENTED SCUTELLARIA BAICALENSIS EXTRACT, FERMENTED SALVIA MILTIORRHIZA EXTRACT AND FERMENTED DRYPTERIS CCRASSIRHIZOMA EXTRACT

(71) Applicant: Sooy-K Co., LTD., Seongnam-si (KR)

(72) Inventors: Youn Ok JUNG, Hwaseong-si (KR); Ji Sun CHUNG, Seongnam-si (KR); Kyoung Mo CHUNG, Seoul (KR); Seo A JUNG, Seongnam-si (KR); Ga Hyeon SONG, Seongnam-si (KR)

(21) Appl. No.: 18/193,209

(22) Filed: Mar. 30, 2023

(30) Foreign Application Priority Data
Mar. 3, 2023 (KR) 10-2023-0028254

(51) Int. Cl. A61K 8/9789; A23L 29/00; A61K 8/9741

(52) U.S. Cl. CPC A61K 8/9789; A23L 29/065; A61K 8/9741; A61K 2800/10; A61K 2800/524; A61K 2800/70; A61K 2800/85

(57) ABSTRACT

Proposed is a composition having antibacterial or antifungal activity, which includes a fermented extract of Scutellaria baicalensis, a fermented extract of Salvia miltiorrhiza, and a fermented extract of Dryopteris crassirhizoma, which, being comprised of natural ingredients, is safe for the human body, and can be effectively used as a natural preservative for foods and cosmetics due to its excellent antibacterial activity.

### TECHNICAL FIELD

The present invention relates to a composition having antibacterial or antifungal activity, which includes a fermented extract of Scutellaria baicalensis, a fermented extract of Salvia miltiorrhiza, and a fermented extract of Dryopteris crassirhizoma, which, being comprised of natural ingredients, is safe for the human body, and has less irritation and excellent antibacterial efficacy, and thus, can be effectively used in foods and cosmetic industries.

### BACKGROUND ART

In the cosmetics and food development process, the use of product keywords such as “vegan”, “cruelty free”, “natural ingredients”, and “preservative-free”, which refer to products that do not require animal experiments and do not contain animal ingredients, is steadily on the increase. In addition to this, with the increasing demand for vegans and natural products as reflected in sales, there has been a remarkable growth in recent years as in the global market size. According to the “Global Organic Personal Care Products Market Status and Forecast” data announced by the Biotechnology Policy Research Center (Korea), the global organic personal care product market grew from USD 8.36 billion in 2013 to USD 15.69 billion in 2020, with an annual growth rate of 9.3%. Due to awareness of the harmful components of synthetic raw materials included in existing personal care products, most consumers tend to prefer organic skin care products.

Recently, with a growing interest in skin safety, many natural cosmetics or paraben-free cosmetics having superiority in skin safety have been developed. This has led to a tendency to use natural ingredients rather than synthetic ingredients whose harmfulness has been proven, and the market size of organic cosmetics and natural cosmetics is gradually increasing. Reviewing the development trend of preservatives, there has been almost no development of new preservatives, and the trend has been progressing in the direction of enhancing the preservative power in the form of a mixture of preservatives that have been used. Considering the possibility of contamination of various microorganisms, the emergence of resistant bacteria, the factors inhibiting preservative actions, etc. encountered during the use of cosmetics, there has been a trend to use several types of preservatives instead of a single preservatives and mix them to produce a synergistic effect. With the emergence of well-being trends at home and abroad, attempts to reduce the proportion of chemical raw materials in cosmetics have been continued. Accordingly, in the cosmetics industry, a number of studies are required for the use of natural antibacterial materials as preservatives to replace chemical preservatives, and these experiments are underway.

“Scutellaria baicalensis”, which is also known as “Chinese skullcap”, belongs to the family Labiatae, and its root has been used as a medicine. It is called ‘yellow gold’ because the color of its root is yellow, and the root contains baicalein, baicalin, wogonin, wogonoside, etc., which are known to be effective in antibacterial, anticancer, and antiinflammatory actions.

“Salvia miltiorrhiza”, which is also known as red sage, Chinese sage, red danshen, or Da Hong Pao, is a perennial plant belonging to the family Labiatae. It is called ‘danshen’ because its root is red. It contains ingredients such as tanshinone, miltinon, and tansinol, and is thus known to have cholesterol-lowering, anti-inflammatory, and antibacterial actions. Although there are differences depending on the species, it has been reported that “Salvia miltiorrhiza” shows excellent antibacterial effects against Gram-positive bacteria.

“Dryopteris ccrassirhizoma”, which is also known as thick stemmed wood fern and aspidium fern, is a perennial plant belonging to the family Aspidiaceae. It is known to have a filmaron's tapeworm control effect, a plavaspidic acid's antibacterial action against Streptococcus mutans, and contains flavaonoid-based components such as baicalin and wogonin.

Accordingly, the present inventors prepared a composition having antibacterial or antifungal activity by fermenting and mixing Scutellaria baicalensis, Salvia miltiorrhiza, and Dryopteris crassirhizoma, and have confirmed that the fermentation mixture have a superior effect compared to the existing natural or synthetic preservatives.

### PRIOR ART DOCUMENTS

(Patent Document 001) KR Patent No. 10-2269881

### SUMMARY

An object to solve in the present invention is to provide a composition having antibacterial or antifungal activity having excellent antibacterial activity.

In order to achieve the technical object above, in an embodiment of the present invention, there is provided a composition having antibacterial or antifungal activity, which includes a fermented extract of Scutellaria baicalensis, a fermented extract of Salvia miltiorrhiza, and a fermented extract of Dryopteris crassirhizoma.

In a specific embodiment, the fermented extract may be fermented by solid fermentation.

In a specific embodiment, the fermented extract may be fermented by any one of bacteria of the genus Lactobacillus and fungi of the genus Saccharomyces.

In a specific embodiment, the fermented extract may be fermented by any one of Lactobacillus casei (L. casei) and Saccharomyces cerevisiae (S. cerevisiae).

In a specific embodiment, the fermented extract may be fermented at a temperature between 20° C. and 40° C.

In a specific embodiment, the fermented extract may be fermented under the condition where the amount of water added is between 40% to 70%.

In a specific embodiment, the composition may be one in which the weight mixing ratio between the fermented extract Scutellaria baicalensis: the fermented extract of Salvia miltiorrhiza: the fermented extract of Dryopteris crassirhizoma is 1:0.5 to 2:0.5 to 1.

In a specific embodiment, the composition may have antibacterial activity against Escherichia coli, Staphylococcus aureus, or Pseudomonas aeruginosa.

In a specific embodiment, the composition may have antifungal activity against Candida albicans or Aspergillus niger.

In a specific embodiment, the composition may be used in one or more selected from the group consisting of cosmetics, skin cleaners, hair cleaners, vaginal cleaners, bathroom cleaners, mouthwashes, kitchen cleaners, cosmetic preservatives, food preservatives, pharmaceutical preservatives, food additives, and feed additives.

Additionally, in another aspect of the present invention, there may be provided a method for preparing a composition having antibacterial or antifungal activity, which includes: fermenting each of Scutellaria baicalensis, Salvia miltiorrhiza, and Dryopteris crassirhizoma by Lactobacillus casei (L. casei) and Saccharomyces cerevisiae (S. cerevisiae) and obtaining fermented extracts thereof; mixing the fermented extracts, diluting the mixture in sterile water, and treating the resultant with ultrasonic waves at 40° C. for 20 to 30 minutes; and filtering the mixed fermented extract.

In a specific embodiment, the Scutellaria baicalensis, Salvia miltiorrhiza, and Dryopteris crassirhizoma may be fermented in a solid state.

In a specific embodiment, the composition may be one in which the weight mixing ratio between the fermented extract Scutellaria baicalensis: the fermented extract of Salvia miltiorrhiza: the fermented extract of Dryopteris crassirhizoma is 1:0.5 to 2:0.5 to 1.

In a specific embodiment, the fermented extracts may be fermented by solid fermentation under the condition where the amount of water added at a temperature between 20° C. and 40° C. is 40% to 70%.

### Advantageous Effects of the Invention

The composition having antibacterial or antifungal activity, which includes a fermented extract of Scutellaria baicalensis, a fermented extract of Salvia miltiorrhiza, and a fermented extract of Dryopteris crassirhizoma, prepared according to an embodiment of the present invention can be effectively utilized as a natural preservative.

The effects of the present invention are not limited to the effects described above, and should be understood to include all effects that can be inferred from the description of the present invention or the constitutions of the invention described in the claims.

### BRIEF DESCRIPTION OF DRAWINGS

FIG. 1 shows the changes in active ingredients (i.e., baicalin, baicalein, and wogonin) before and after fermentation. of Scutellaria baicalensis.

FIG. 2 shows the change in the active ingredient (i.e., salvianolic acid A) before and after fermentation of Salvia miltiorrhiza.

FIG. 3A shows the peak of flavaspicid acid AP in the Dryopteris crassirhizoma extract before fermentation.

FIG. 3B shows the peak of flavaspicid acid AP in the Dryopteris crassirhizoma extract after fermentation, which was obtained after the fermentation under the conditions of S. cerevisiae, 30° C., and 60%.

FIG. 3C shows the peak of flavaspicid acid PB in the Dryopteris crassirhizoma extract before fermentation.

FIG. 3D shows the peak of flavaspicid acid PB in the Dryopteris crassirhizoma extract after fermentation, which was obtained after the fermentation under the conditions of S. cerevisiae, 30° C., and 60%.

FIG. 3E shows the peak of flavaspicid acid AB in the Dryopteris crassirhizoma extract before fermentation.

FIG. 3F shows the peak of flavaspicid acid AB in the Dryopteris crassirhizoma extract after fermentation, which was obtained after the fermentation under the conditions of S. cerevisiae, 30° C., and 60%.

FIG. 3G shows the peak of flavaspicid acid BB in the Dryopteris crassirhizoma extract before fermentation.

FIG. 3H shows the peak of flavaspicid acid BB in the Dryopteris crassirhizoma extract after fermentation, which was obtained after the fermentation under the conditions of S. cerevisiae, 30° C., and 60%.

FIG. 4 shows microscopic images of fermented extracts fermented by Bacillus subtilis after 7 days of fermentation.

FIG. 5 shows microscopic images of fermented extracts fermented by Lactobacillus casei after 7 days of fermentation.

FIG. 6 shows microscopic images of fermented extracts fermented by Saccharomyces cerevisiae after 7 days of fermentation.

FIG. 7 shows microscopic images of fermented extracts fermented by Aspergillus niger after 7 days of fermentation.

FIG. 8 shows the results of antibacterial experiments using Scutellaria baicalensis.

FIG. 9 shows the results of antibacterial experiments using Salvia miltiorrhiza.

FIG. 10 shows the results of antibacterial experiments using Dryopteris crassirhizoma.

FIG. 11 shows the results of antibacterial activity of a 60% fermented extract of Scutellaria baicalensis by L. casei (LC) at 37° C.

FIG. 12 shows the results of antibacterial activity of a 50% fermented extract of Scutellaria baicalensis by S. cerevisiae (SC) at 37° C.

FIG. 13 shows the results of antibacterial activity of a fermented extract of Salvia miltiorrhiza by S. cerevisiae (SC) at 30° C.

FIG. 14 shows the results of antibacterial activity of a fermented extract of Salvia miltiorrhiza by S. cerevisiae (SC) at 37° C.

FIG. 15 shows the results of antibacterial activity of a 60% fermented extract of Dryopteris crassirhizoma by L. casei (LC) at 30° C.

FIG. 16 shows the results of antibacterial activity of a 60% fermented extract of Dryopteris crassirhizoma by S. cerevisiae (SC) at 30° C.

FIG. 17 shows the results of antibacterial activity of a complex extract of Scutellaria baicalensis, Salvia miltiorrhiza, and Dryopteris crassirhizoma (Sample No. 16).

FIG. 18 shows the results of antibacterial activity of a complex extract of Scutellaria baicalensis, Salvia miltiorrhiza, and Dryopteris crassirhizoma (Sample No. 20).

FIG. 19 shows the results of antibacterial activity of a complex extract of Scutellaria baicalensis, Salvia miltiorrhiza, and Dryopteris crassirhizoma (Sample No. 24).

FIG. 20 shows the comparison results of antibacterial activity between Anti-Bac-FSB (which is a natural preservative) and antibiotics.

FIG. 21 shows the comparison results of antibacterial activity between Anti-Bac-FSB and commercially available preservatives (which are natural preservatives) and common preservatives.

### DETAILED DESCRIPTION

Hereinafter, the present invention will be described in detail.

According to an aspect of the present invention, there is provided a composition having antibacterial or antifungal activity, which includes a fermented extract of Scutellaria baicalensis, a fermented extract of Salvia miltiorrhiza, and a fermented extract of Dryopteris crassirhizoma.

Fermentation means that microorganisms (e.g., yeast or bacteria) decompose organic compounds by a comprehensive action of the external environment (e.g., temperature, humidity, time, space, etc.) and thereby the components are decomposed from high molecules to low molecules. According to the present invention, when Scutellaria baicalensis, Salvia miltiorrhiza, and Dryopteris crassirhizoma are each fermented with a specific strain, respectively, a fermented product is produced therefrom according to the fermentation conditions. As such, the fermentation results vary depending on various fermentation conditions (e.g., enzymes, temperature, humidity, time, etc.).

In the present invention, the fermented extract may be solid fermentation.

As used herein, the term “solid fermentation” may refer to a type of fermentation technology in which microorganisms inoculated into a solid medium containing low moisture of Aw (water activity) of 0.40 to 0.90 grow on the surface of a medium and then gradually proliferate through the space between the particles inside a substrate to thereby produce a target material.

The fermented extract may be one which is fermented with any one of bacteria of the genus Lactobacillus and fungi of the genus Saccharomyces.

The fermented extract may be one which is fermented with any one of Lactobacillus casei (L. casei) and Saccharomyces cerevisiae (S. cerevisiae).

The fermented extract may be one which is fermented at a temperature between 20° C. and 40° C.

The fermented extract may be one which is fermented under the condition where the amount of water added is between 40% to 70%.

The composition may be one in which the weight mixing ratio between the fermented extract Scutellaria baicalensis: the fermented extract of Salvia miltiorrhiza: the fermented extract of Dryopteris crassirhizoma is 1:0.5 to 2:0.5 to 1. More preferably, the weight mixing ratio may be 1:1:0.5.

In the present invention, antibacterial and antifungal activity was not shown when the fermented extracts were used alone or partially mixed; however, improved antibacterial and antifungal activity can be obtained by preparing a mixture of the fermented extracts at a specific mixing ratio.

The composition may have antibacterial activity against Escherichia coli, Staphylococcus aureus, or Pseudomonas aeruginosa.

The composition may have antifungal activity against Candida albicans or Aspergillus niger.

As used herein, the term “antibacterial activity” may refer to inhibition of the activity or growth of pathogenic bacteria, and further refer to killing of pathogenic bacteria. However, the term is not limited thereto, and antibacterial activity as used herein may refer to inhibitory activity of all phenomena caused by pathogenic bacteria.

As used herein, the term “antibacterial composition” refers to a preparation that is provided to a subject in the form of a drug and can kill bacteria, and is a generic term for preservatives, bactericides, antibiotics, and antibacterial agents.

The composition may be used in one or more selected from the group consisting of cosmetics, skin cleaners, hair cleaners, vaginal cleaners, bathroom cleaners, mouthwashes, kitchen cleaners, cosmetic preservatives, food preservatives, pharmaceutical preservatives, food additives, and feed additives.

In the present invention, when the composition is used as a material for a preservative, the products to which it can be applied are not particularly limited, and specifically may be applied to cosmetics, foods, pharmaceuticals, etc. In this case, the composition may be included in an amount of 0.00001 wt % to 50 wt % based on the total weight of the preservative, and may be included in a concentration of 1 μg/mL to 10,000 μg/mL. Additionally, the preservative may further include other known materials having antibacterial activity or preservative activity in addition to the composition.

Additionally, according to the present invention, there is provided a method for preparing a composition having antibacterial or antifungal activity, which includes: fermenting each of Scutellaria baicalensis, Salvia miltiorrhiza, and Dryopteris crassirhizoma by Lactobacillus casei (L. casei) and Saccharomyces cerevisiae (S. cerevisiae) and obtaining fermented extracts thereof; mixing the fermented extracts, diluting the mixture in sterile water, and treating the resultant with ultrasonic waves at 40° C. for 20 to 30 minutes; and filtering the mixed fermented extract.

The Scutellaria baicalensis, Salvia miltiorrhiza, and Dryopteris crassirhizoma may be fermented in a solid state.

The composition may be one in which the weight mixing ratio between the fermented extract Scutellaria baicalensis: the fermented extract of Salvia miltiorrhiza: the fermented extract of Dryopteris crassirhizoma is 1:0.5 to 2:0.5 to 1.

The fermented extracts may be fermented by solid fermentation under the condition where the amount of water added at a temperature between 20° C. and 40° C. is 40% to 70%.

### EXPERIMENTAL EXAMPLES

### 1. Preparation of Fermentation Strains

As strains used for fermentation, Bacillus subtilis (B. subtilis), Lactobacillus casei (L. casei), and Saccharomyces cerevisiae (S. cerevisiae) were provided by Professor Geun Kim of University of Suwon (Korea) and used in the experiment, and Aspergillus oryzae (Asp. oryzae) is a strain which was isolated from yeast and identified by the Korea Culture Center of Microorganisms through 18S rRNA analysis (Table 1). B. subtilis was cultured in nutrient agar (Difco Co., USA), L. casei in MRS agar (Difco Co., USA), S. cerevisiae in yeast malt agar (Difco Co., USA), and Asp. oryzae in potato dextrose agar (Difco Co., USA), respectively, and used in the experiment.

TABLE 1
Strains used for fermentation

| Strains | Sources |
|---|---|
| Bacillus subtilis | doenjang |
| Lactobacillus casei | cheese |
| Saccharomyces cerevisiae | grapes |
| Aspergillus oryzae | fermented soybeans |

### 2. Conditions for High-Performance Liquid Chromatography (HPLC) Analysis

The high-performance liquid chromatography (HPLC) analysis of the Scutellaria baicalensis extract was performed by HPLC (Nexera lite, Shimadzu, Japan) using a Mightysil RP-18 GP column (4.6 mm×250 mm (5 m), Kanto, TOKYO). After adjusting the flow rate to 1.0 mL/min using 0.1% phosphoric acid in water (A) and acetonitrile (B) as the mobile phase, 10 μL was injected thereto, and the extract was separated using a gradient elution. The temperature of the column was maintained at 30° C., and the extract was analyzed by measuring at 275 nm using a UV-visible detector (Shimadzu, Japan).

TABLE 2
Conditions for HPLC analysis of Scutellaria baicalensis extract

| Instruments | Condition |
|---|---|
| Column | Mightysil RP-18GP 250-4.6 (5 μm) |
| Mobile phase | solvent A: 0.1% phosphoric acid in water |
| | solvent B: acetonitrile |
| | <gradient> |
| | 20-30% B (0-10 min) |
| | 30-33% B (10-20 min) |
| | 33-60% B (20-30 min) |
| | 60-100% B (30-35 min) |
| | 100% B (35-40 min) |
| | 100%-20% B (40-40.1 min) |
| | 20% B (40.1-45 min) post time |
| Detector | 275 nm |
| Flow rate | 1.0 mL/min |
| Column temperature | 30° C. |
| Injection volume | 10 μL |

The high-performance liquid chromatography (HPLC) analysis of the Salvia miltiorrhiza extract was performed by HPLC (Nexera lite, Shimadzu, Japan) using a Mightysil RP-18 GP column (4.6 mm×250 mm (5 μm), Kanto, Tokyo). After adjusting the flow rate to 1.0 mL/min using 75% methanol as the mobile phase, 10 μL was injected thereto, and the extract was separated using a gradient elution. The temperature of the column was maintained at 30° C., and the extract was analyzed by measuring at 275 nm using a UV-visible detector (Shimadzu, Japan) (Table 3).

TABLE 3
Conditions for HPLC Analysis of Salvia miltiorrhiza extract

| Instrument | Condition |
|---|---|
| Column | Mightysil RP-18GP 250-4.6 (5 μm) |
| Mobile phase | 75% MeOH (isocratic elution) |
| Detector | 270 nm |
| Flow rate | 1.0 mL/min |
| Column temperature | 30° C. |
| Injection volume | 10 μL |

3. Fermentation Conditions of Scutellaria baicalensis, Salvia Miltiorrhiza, and Dryopteris crassirhizoma Extracts

10 g each of raw materials of Scutellaria baicalensis, Salvia miltiorrhiza, and Dryopteris crassirhizoma was sterilized at 121° C. and 1.5 atm for 30 minutes under the condition where the amount of initial water addition was 40%, 50%, or 60%. After allowing the sterilized raw materials to be cooled at room temperature, the strain culture solutions were dispensed in the amount of 1 mL each, and fermented according to the culture temperature (Table 4).

TABLE 4
Conditions for solid fermentation

| Strains | Temperature (° C.) | Moisture content (%) |
|---|---|---|
| Control Group | — | 40 |
| | | 50 |
| | | 60 |
| B. subtilis | 30 | 50 |
| | | 60 |
| | 37 | 50 |
| | | 60 |
| L. casei | 30 | 50 |
| | | 60 |
| | 37 | 50 |
| | | 60 |
| S. cerevisiae | 30 | 50 |
| | | 60 |
| | 37 | 50 |
| | | 60 |
| Asp. oryzae | 25 | 40 |
| | | 50 |
| | 30 | 40 |
| | | 50 |

### 4. Evaluation of Antibacterial Activity (Disc Diffusion Method)

As the strains used for antibacterial activity experiments, Gram-positive bacteria Staphylococcus aureus (S. aureus, KCTC 1916), Gram-negative Escherichia coli (E. coli, KCTC 2571), Pseudomonas aeruginosa (P. aeruginosa, KCTC 2513), Candida albicans (C. albicans, KCTC 7965), and Aspergillus niger (A. niger, KCTC 6196), which were purchased from the Korean collection for Type Cultures (KCTC), were stored and used (Table 5).

The inhibitory zones of the extracts were measured by a test method of antibacterial activity to determine the presence or absence of antibacterial activity and to compare the degree of effectiveness of the extracts against the proliferation of bacteria. The antibacterial activity was measured by the disc diffusion method using a paper disc (size: 8 mm, Advantec, Toyo Roshi Kaisha, Ltd., Tokyo, Japan). The strains were each inoculated in an amount of one platinum loop, diluted in 4 mL of sterile water, mixed with a sterilized medium, and the mixture was poured into a prepared plate to be hardened. A 3% extract each was filtered (pore size: 0.4 μm, Advantec MFS, Inc., Tokyo, Japan), and 40 μL was taken and allowed to be absorbed onto a paper disc. After culturing for 24 hours at an appropriate temperature for each strain, the size of the transparent ring formed around the paper disc was measured.

TABLE 5
List of strains for antibacterial experiment

| Strain name (Country name)/ | | |
|---|---|---|
| Medium | Strain No. | BioSafety Level |
| Escherichia coli/ | KCTC2571 | Level 1 |
| Nutrient Agar, TSA | | |
| Staphylococcus aureus/ | KCTC1916 | |
| LB, TSA | | |
| Pseudomonas aeruginosa/ | KCTC2513 | |
| TSA | | |
| Candida albicans/ | KCTC7965 | Level 2 |
| YM, SDA Agar | | |
| Aspergillus brasiliensis | KCTC6196 | Level 4 |
| (niger)/Potato Dextrose, | | |
| YM, SDA Agar | | |

### 5. Preparation of Fermentation Complex

After fermenting Scutellaria baicalensis, Salvia miltiorrhiza, and Dryopteris crassirhizoma, fermentation complexes were prepared according to the mixing ratios by selecting fermentation conditions under which excellent antibacterial activity was shown (Table 6).

TABLE 6
Fermentation conditions showing excellent antibacterial
activity according to species.

| Species | Strains and Treatment Methods | |
|---|---|---|
| Scutellaria | L. casei, 37° C., 60%, | S. cerevisiae, 37° C., 50%, |
| baicalensis | solid fermentation | solid fermentation |
| Salvia | S. cerevisiae, 30° C., 50%, | S. cerevisiae, 37° C., 50%, |
| miltiorrhiza | solid fermentation | solid fermentation |
| Dryopteris | L. casei, 30° C., 60%, | S. cerevisiae, 30° C., 60%, |
| crassirhizoma | solid fermentation | solid fermentation |

### <Analysis of Results>

1. Analysis of Scutellaria baicalensis, Salvia miltiorrhiza, and Dryopteris crassirhizoma Extracts by HPLC
1) Analysis of Scutellaria baicalensis Extract by HPLC

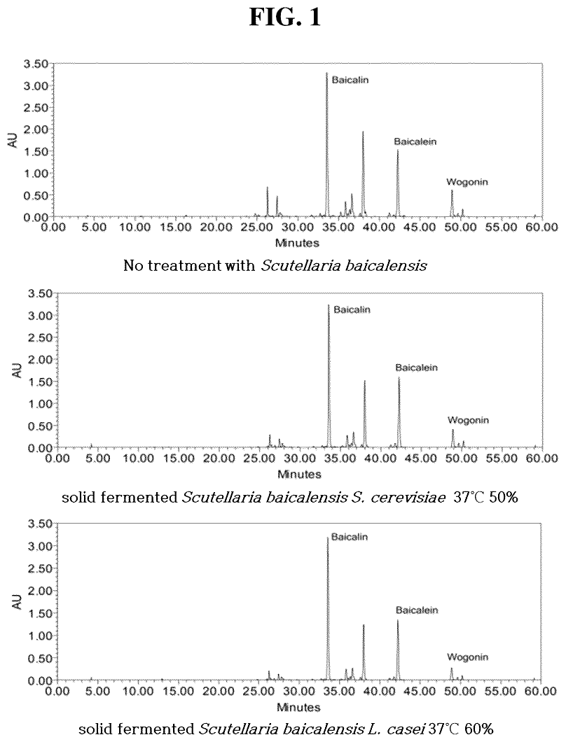

The changes in the contents of baicalin, baicalein, and wogonin, which are active components present in Scutellaria baicalensis, before and after fermentation were confirmed, and as a result of qualitative/quantitative analysis, it was found that the contents of these components present in Scutellaria baicalensis before fermentation were as follows: baicalin (101.57 mg/g), baicalein (28.26 mg/g), and wogonin (5.33 mg/g) (FIG. 1).

As a result of performing fermentation while varying the fermentation microorganism, temperature, and amount of water addition (%) before inoculation, it was found that the contents of these components under the conditions of S. cerevisiae, 37° C., and 50% were baicalin (94.31 mg/g), baicalein (30.41 mg/g), and wogonin (3.57 mg/g); and that the contents of these components under the conditions of L. casei, 37° C., and 60% were baicalin (88.62 mg/g), baicalein (25.36 mg/g), and wogonin (2.44 mg/g). It was found that the increase in baicalein after fermentation of Scutellaria baicalensis under the conditions of S. cerevisiae, 37° C., and 50% compared to before the fermentation of Scutellaria baicalensis, and it is interpreted that this might be due to the bioconversion of one molecule of glucose in baicalin, which is in the form of a glycoside, into baicalein by hydrolysis.

2) Analysis of Salvia miltiorrhiza Extract by HPLC

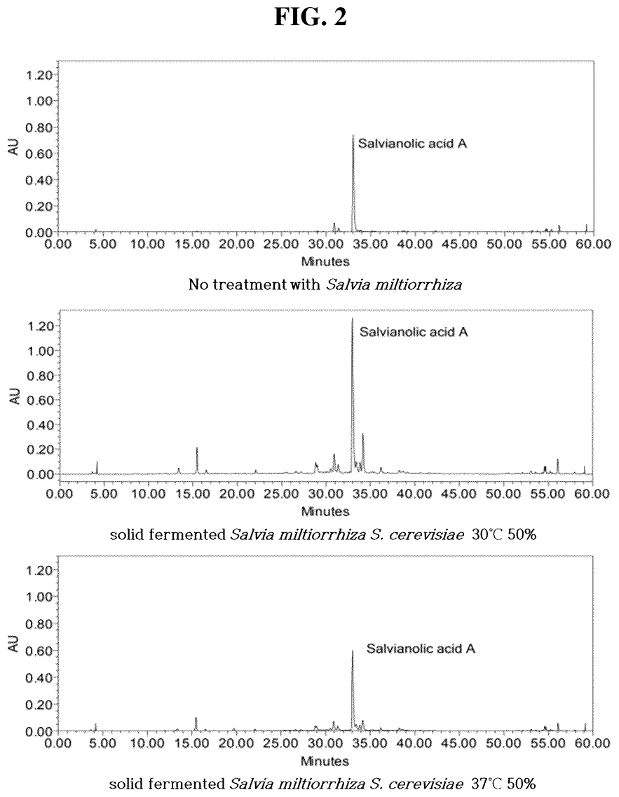

The changes in the content of salvianolic acid A, which is the active component present in Salvia miltiorrhiza, before and after fermentation were confirmed, and it was analyzed that the content of salvianolic acid A present in Salvia miltiorrhiza before fermentation was shown to be 1.82 mg/g (FIG. 2).

As a result of performing fermentation by varying the temperature while using the fermentation microorganism (i.e., S. cerevisiae), it was found that the content of salvianolic acid A was increased to 5.70 mg/g under the fermentation condition of 30° C., whereas the content of salvianolic acid A was decreased to 1.41 mg/g under the fermentation condition of 37° C.

3) Analysis of Dryopteris crassirhizoma Extract by HPLC

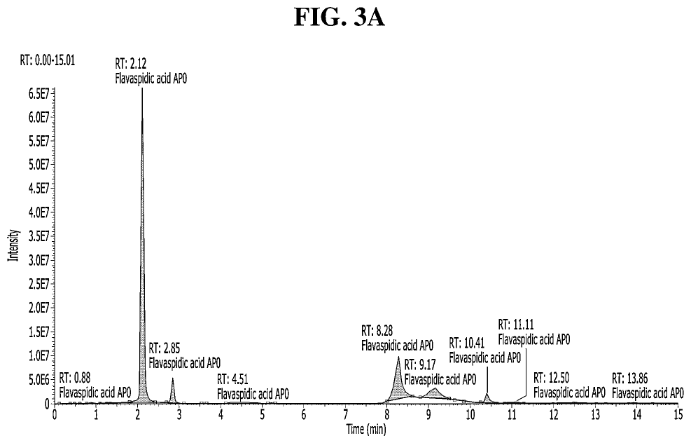

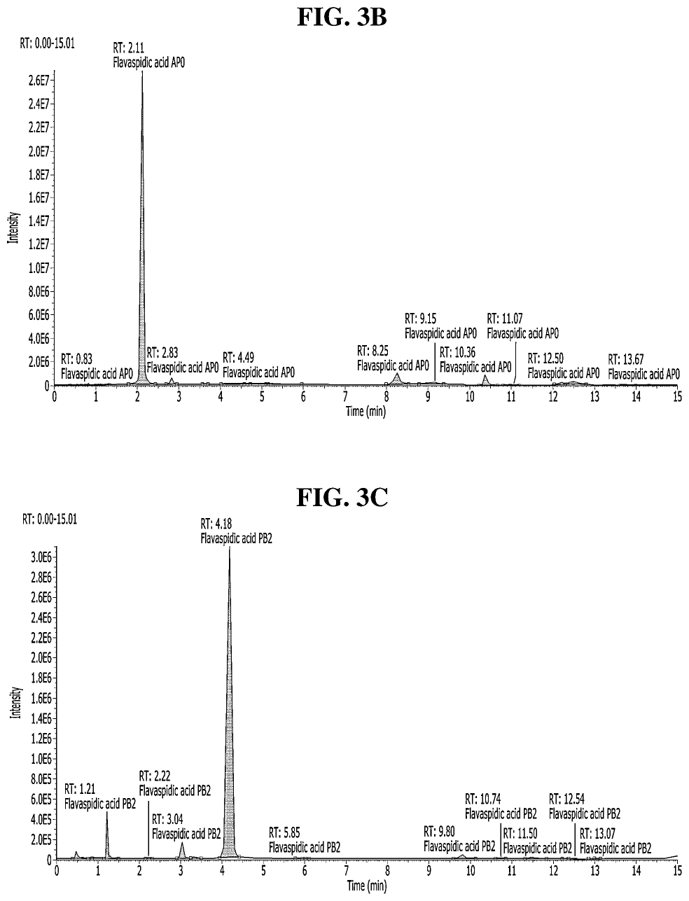

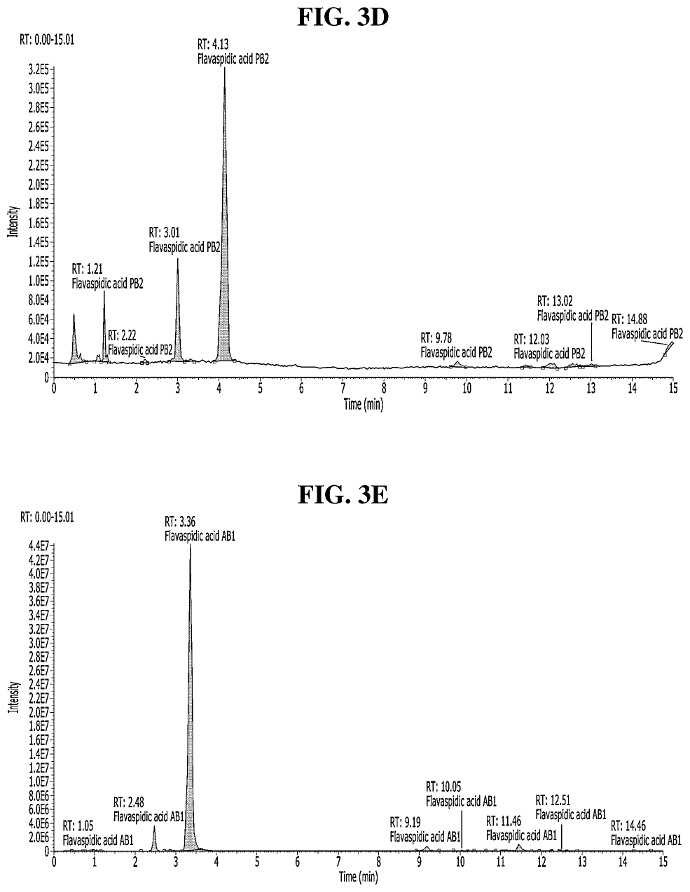

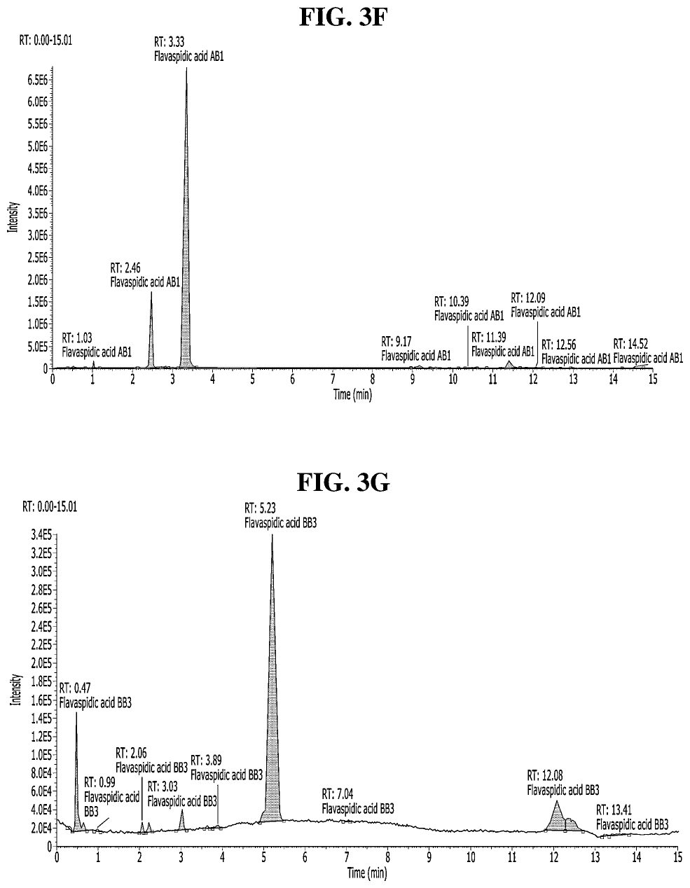

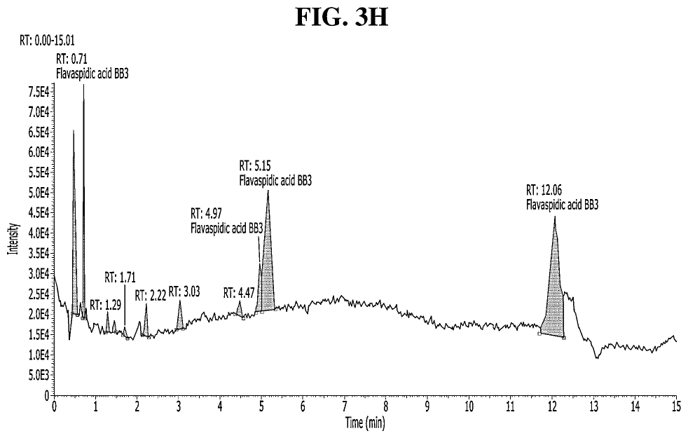

Through the Dryopteris crassirhizoma extract and a fermented extract thereof, it was found that the 19 phenolic compounds and 4 standard products (baicalin, baicalein, wogonin, and salvianolic acid A) reported as components derived from Dryopteris crassirhizoma were also not detected. The excellent antibacterial activities of phloroglucinol-based derivatives (flavaspidic acid AP, flavaspidic acid PB, flavaspidic AB, and flavaspidic acid BB) (Table 7) and quinolone-based derivatives were confirmed by referring to the thesis documents and patents reported for re-selection of efficacy components. However, since the two reported derivatives are both components that were commercially unavailable and thus the quantitative analysis of these components by LC or LC-MS were impossible, the result was derived by determining only the contents of the active ingredients in the extracts through LC-MS qualitative analysis. The LC-MS-Scan results revealed that in both the Dryopteris crassirhizoma extract before fermentation and the Dryopteris crassirhizoma extract after fermentation, which was obtained after the fermentation under the conditions of S. cerevisiae, 30° C., and 60%, there were 4 types of flavaspidic acid derivatives of the phloroglucinol family with molecular weights of 404, 432, 418, and 446, and the peaks of materials, which were presumed to be flavaspicid acid AP, flavaspicid acid PB, flavaspicid acid AB, and flavaspicid acid BB, were discovered (FIGS. 3A-3H).

TABLE 7
Names of active components of Dryopteris crassirhizoma
and molecular weights thereof

| | | Chemical | Molecular |
|---|---|---|---|
| Names of Components | | Formula | Weight |
| Phloroglucinol | flavaspidic acid AP | C₂₁H₂₄O₈ | 404 |
| | flavaspidic acid PB | C₂₃H₂₈O₈ | 432 |
| | flavaspidic acid AB | C₂₂H₂₆O₈ | 418 |
| | flavaspidic acid BB | C₂₄H₃₀O₈ | 446 |

### 2. Conditions for Cultivation of Strains and Evaluation of Antibacterial Activity

Scutellaria baicalensis, Salvia miltiorrhiza, and Dryopteris crassirhizoma were subjected to solid fermentation and the states of these strains were confirmed.

1) State of Bacillus subtilis after 7 Days of Solid Fermentation

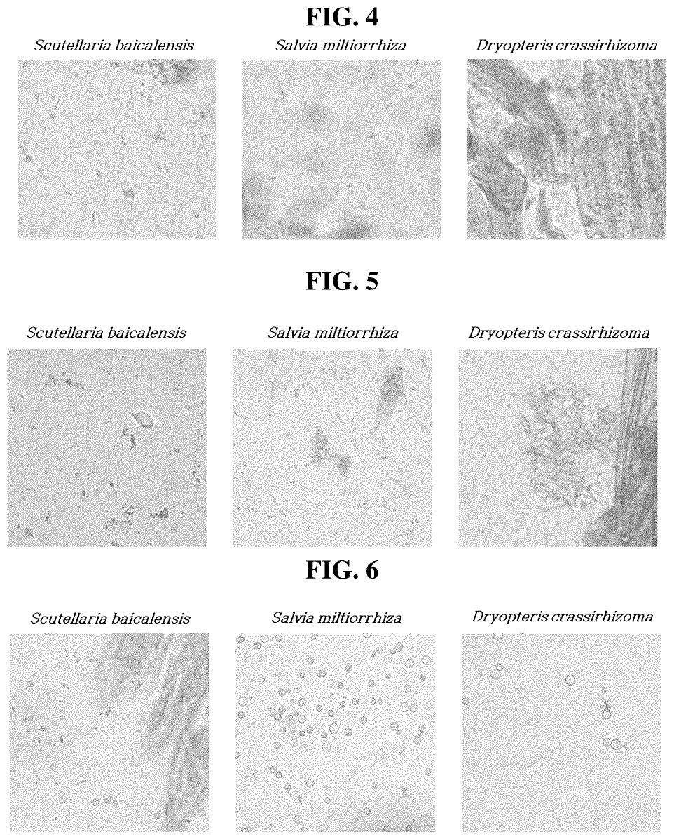

Before the introduction of the strain, it was confirmed that the density of Bacillus subtilis was high, but after fermentation, the density observed under a microscope was very low, and in particular, it was found that almost no Dryopteris crassirhizoma survived (FIG. 4).

2) State of Lactobacillus casei after 7 Days of Solid Fermentation

As a result of performing solid fermentation using Lactobacillus casei for 7 days, it was confirmed that the strain survived to some extent in Salvia miltiorrhiza and Dryopteris crassirhizoma, but there was a distinct decrease in the case of Scutellaria baicalensis. (FIG. 5).

3) State of Saccharomyces cerevisiae after 7 Days of Solid Fermentation

As a result of solid fermentation using Saccharomyces cerevisiae for 7 days, many yeasts still survived in Salvia miltiorrhiza, but most were shown to be dead in of Scutellaria baicalensis and Dryopteris crassirhizoma (FIG. 6).

4) State of Aspergillus oryzae after 7 Days of Solid Fermentation

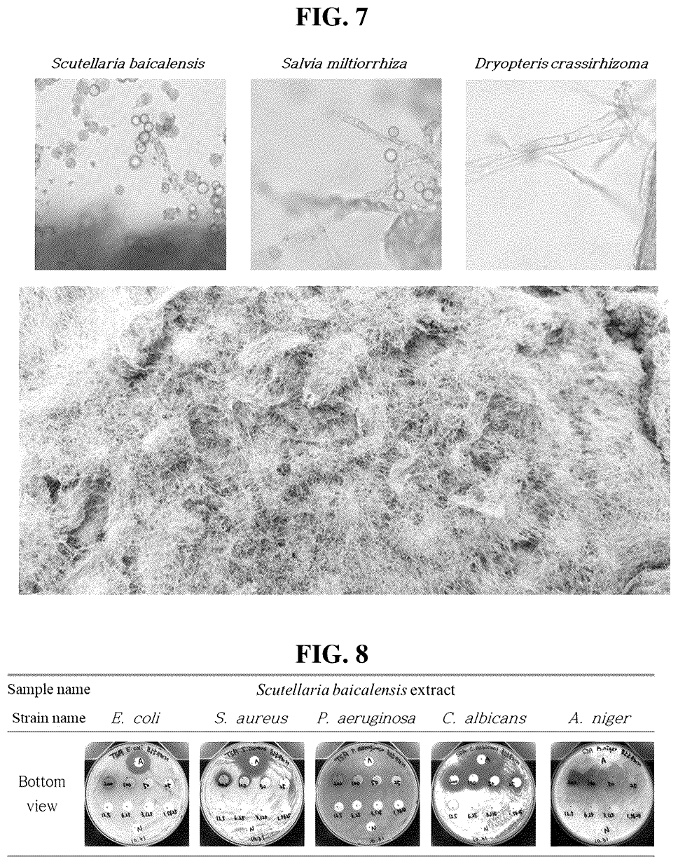

Fungi are strains which are not used in liquid fermentation but can be used in solid fermentation. As a result of performing fermentation using Aspergillus oryzae in solid fermentation, it was confirmed that the strain grew well in all of the groups of treated with Scutellaria baicalensis, Salvia miltiorrhiza, and Dryopteris crassirhizoma. (FIG. 7).

### 3. Conditions for Cultivation of Strains and Evaluation of Antibacterial Activity

1) Antibacterial Activity of Scutellaria baicalensis Extract

As a result of evaluation of antibacterial activity of a Scutellaria baicalensis extract according to its treatment at various concentrations (1.56 mg/mL to 200 mg/mL), in terms of the inhibition of the growth of Candida albicans, the size of the clear zone was shown to be 9.7 mm at 12.5 mg/mL, 14.2 mm at 25 mg/mL, 15.7 mm at 50 mg/mL, 18.4 mm at 100 mg/mL, and 24.3 mm at 200 mg/mL. Through the results above, it was confirmed that the activity of Candida albicans was shown to be high in a concentration-dependent manner, and from the result that the clear zone was shown to be large starting from the concentration of 50 mg/mL, it can be seen that the minimum concentration of the Scutellaria baicalensis extract against the activity of Candida albicans is 50 mg/mL.

In Staphylococcus aureus, the clear zone began to appear from the concentration of 50 mg/mL, and the size of the clear zone was shown to be 9.2 mm at 50 mg/mL, 11.8 mm at 100 mg/mL, and 14.8 mm at 200 mg/mL. From these results, it was found that the minimum concentration of the Scutellaria baicalensis extract for inhibition of Staphylococcus is 200 mg/mL.

In Escherichia coli (E. coli) and Pseudomonas aeruginosa (P. aeruginosa), the clear zones were shown to be weak. In E. coli, the size of the clear zone was shown to be 9.8 mm at 100 mg/mL and 12.5 mm at 200 mg/mL, thus confirming that the clear zone began to appear from the minimum concentration of 200 mg/mL. In P. aeruginosa, overall, the clear zones were shown to be weak, and among them, the clear zones were shown only at the concentration of 200 mg/mL and they were shown to be weak having a size of 9.0 mm. Additionally, in Aspergillus niger, no clear zones were observed within the experimental criteria.

That is, as a result of performing antibacterial experiments using a Scutellaria baicalensis extract, it was found that the largest clear zones were shown in Candida albicans, and more than a certain level of effect was also shown Staphylococcus, but in E. coli and P. aeruginosa, the clear zones were shown only at the concentration of 200 mg/mL and were shown to be weak (FIG. 8).

TABLE 8
Antibacterial experiment using Scutellaria baicalensis extract

| | Conc. (mg) | Clear Zone (ø, mm) | | | | |
|---|---|---|---|---|---|---|
| Paper | Stock | | S. | P. | C. | A. |
| Disc | Conc. | E. coli | aureus | aeruginosa | albicans | niger |
| No treatment | 200 | 12.5 | 14.8 | 9.0 | 24.3 | N.A. |
| with | 100 | 9.8 | 11.8 | N.A. | 18.4 | N.A. |
| Scutellaria | 50 | N.A. | 9.2 | N.A. | 15.7 | N.A. |
| baicalensis | 25 | N.A. | N.A. | N.A. | 14.2 | N.A. |
| | 12.5 | N.A. | N.A. | N.A. | 9.7 | N.A. |
| | 6.25 | N.A. | N.A. | N.A. | N.A. | N.A. |
| | 3.125 | N.A. | N.A. | N.A. | N.A. | N.A. |
| | 1.5626 | N.A. | N.A. | N.A. | N.A. | N.A. |
| Control | Antibiotic | 25.6 | 22.8 | N.A. | 26.3 | N.A. |
| Group | Negative | N.A. | N.A. | N.A. | N.A. | N.A. |
| | Control | | | | | |

Antibiotic: ketoconazole (100 μg/mL), tetracycline (50 μg/mL)
Negative control: distilled water (diluent)
N.A.: no activity, no influence

2) Antibacterial Activity of Salvia miltiorrhiza Extract

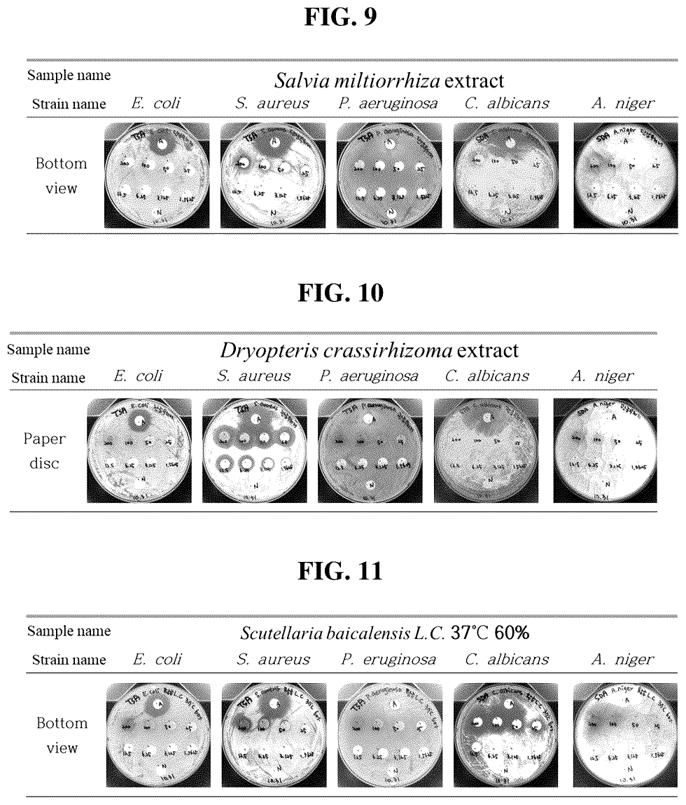

As a result of confirming the antibacterial activity of a Salvia miltiorrhiza extract by the disc diffusion method according to its treatment at various concentrations (1.56 mg/mL to 200 mg/mL), only the clear zones against Staphylococcus aureus (S. aureus) were found. The antibacterial effect against S. aureus began to appear from the concentration of 25 mg/mL, and the size of the clear zones was 11.2 mm at 25 mg/mL, 11.6 mm at 50 mg/mL, 11.5 mm at 100 mg/mL, and 13.8 mm at 200 mg/mL, which was shown to be largest. The Salvia miltiorrhiza extract showed no antibacterial effect at all in other strains. That is, it was found that the Salvia miltiorrhiza extract was effective only in Staphylococcus aureus (FIG. 9).

TABLE 9
Antibacterial experiment using Salvia miltiorrhiza extract

| | Conc. (mg) | Clear Zone (ø, mm) | | | | |
|---|---|---|---|---|---|---|
| Paper | Stock | | S. | P. | C. | A. |
| Disc | Conc. | E. coli | aureus | aeruginosa | albicans | niger |
| No treatment | 200 | N.A. | 13.8 | N.A. | N.A. | N.A. |
| with Salvia | 100 | N.A. | 11.5 | N.A. | N.A. | N.A. |
| miltiorrhiza | 50 | N.A. | 11.6 | N.A. | N.A. | N.A. |
| | 25 | N.A. | 11.2 | N.A. | N.A. | N.A. |
| | 12.5 | N.A. | N.A. | N.A. | N.A. | N.A. |
| | 6.25 | N.A. | N.A. | N.A. | N.A. | N.A. |
| | 3.125 | N.A. | N.A. | N.A. | N.A. | N.A. |
| | 1.5626 | N.A. | N.A. | N.A. | N.A. | N.A. |
| Control | Antibiotic | 19.6 | 22.8 | 9.0 | 16.2 | N.A. |
| Group | Negative | N.A. | N.A. | N.A. | N.A. | N.A. |
| | Control | | | | | |

Antibiotic: ketoconazole (100 μg/mL), tetracycline (50 μg/mL)
Negative control: distilled water (diluent)
N.A.: no activity, no influence

3) Antibacterial Experiment Using Dryopteris crassirhizoma Extract

As a result of confirming the antibacterial activity of a Dryopteris crassirhizoma extract by the disc diffusion method according to its treatment at various concentrations (1.56 mg/mL to 200 mg/mL), in Staphylococcus aureus, the antibacterial effect against strains was shown to be weak by exhibiting a weak clear zone with a size of 9.2 mm at 1.56 mg/mL, and starting from the concentration of 6.25 mg/mL, the clear zones gradually became wider as the concentration increased, and the concentration at which the clear zones having antibacterial activity were shown to be widest with a size of 20.8 mm at 200 mg/mL. The Dryopteris crassirhizoma extract showed no antibacterial effect at all in other strains. That is, it was found that the Dryopteris crassirhizoma extract was effective in Staphylococcus aureus even at low concentrations, it was not effective in other strains (FIG. 10).

TABLE 10
Antibacterial experiment using Dryopteris crassirhizoma extract

| | Conc. (mg) | Clear Zone (ø, mm) | | | | |
|---|---|---|---|---|---|---|
| Paper | Stock | | S. | P. | C. | A. |
| Disc | Conc. | E. coli | aureus | aeruginosa | albicans | niger |
| No treatment | 200 | N.A. | 20.8 | N.A. | N.A. | N.A. |
| with | 100 | N.A. | 18.6 | N.A. | N.A. | N.A. |
| Dryopteris | 50 | N.A. | 17.2 | N.A. | N.A. | N.A. |
| crassir- | 25 | N.A. | 17.0 | N.A. | N.A. | N.A. |
| hizoma | 12.5 | N.A. | 15.8 | N.A. | N.A. | N.A. |
| | 6.25 | N.A. | 13.5 | N.A. | N.A. | N.A. |
| | 3.125 | N.A. | 11.8 | N.A. | N.A. | N.A. |
| | 1.5626 | N.A. | 9.2 | N.A. | N.A. | N.A. |
| Control | Antibiotic | 18.2 | 23.8 | N.A. | 23.8 | N.A. |
| Group | Negative | N.A. | N.A. | N.A. | N.A. | N.A. |
| | Control | | | | | |

Antibiotic: ketoconazole (100 μg/mL), tetracycline (50 μg/mL)
Negative control: distilled water (diluent)
N.A.: no activity, no influence

In summary, the treatment with the Scutellaria baicalensis extract resulted in the appearance of clear zones, although not significant, in E. coli, P. aeruginosa, and S. aureus; showed no effect against A. niger and showed the widest and broadest clear zones in C. albicans. The treatment with the Salvia miltiorrhiza extract showed no antibacterial activity at all against E. coli, C. albicans, P. aeruginosa, and S. aureus, and clear zones appeared only in S. aureus. The treatment with the Dryopteris crassirhizoma extract showed no antibacterial activity at all against E. coli, C. albicans, P. aeruginosa, and S. aureus, and overall, excellent clear zones appeared in S. aureus.

Accordingly, it was confirmed that the Scutellaria baicalensis extract, Salvia miltiorrhiza extract, and Salvia miltiorrhiza extract were most effective against S. aureus.

4. Evaluation of Antibacterial Activity after Fermentation According to Conditions
1-1) Antibacterial Activity of Scutellaria baicalensis Extract Fermented Under 37° C. and 60% Conditions Using L. casei (L. C.)

As a result of confirming the antibacterial activity of a Scutellaria baicalensis extract fermented under 37° C. and 60% conditions using L. casei (L. C.) by the disc diffusion method according to its treatment at various concentrations (1.56 mg/mL to 200 mg/mL), clear zones began to appear in growth inhibition against E. coli from the concentration of 50 mg/mL, and the size of the clear zone was 11.2 mm in the group treated at the concentration of 200 mg/mL. The growth inhibition against C. albicans began to appear at a low concentration of 12.5 mg/mL and the size of the clear zone became wider as the treatment concentration became higher, thus showing the widest size of 24.2 mm at 200 mg/mL. As for the inhibition against P. aeruginosa, the size of the clear zone was 12.0 mm at 200 mg/mL thus confirming that clear zones appear only at higher concentrations, which indicate that the clear zones were weak. As for the inhibition against S. aureus, clear zones began to appear from the concentration of 50 mg/mL and the size of the clear zone became wider as the treatment concentration became higher, thus showing the size of 14.2 mm at 200 mg/mL. The fermented Scutellaria baicalensis extract showed no effect at all against A. niger regardless of its concentration (FIG. 11).

TABLE 11
Antibacterial activity of Scutellaria baicalensis extract fermented
under 37° C. and 60% conditions using L. casei (L. C.)

| | Conc. (mg) | Clear Zone (ø, mm) | | | | |
|---|---|---|---|---|---|---|
| Paper | Stock | | S. | P. | C. | A. |
| Disc | Conc. | E. coli | aureus | aeruginosa | albicans | niger |
| Scutellaria | 200 | 11.2 | 14.2 | 12.0 | 24.2 | N.A. |
| baicalensis | 100 | 9.7 | 11.3 | N.A. | 22.3 | N.A. |
| casei (L. C.), | 50 | 9.4 | 9.1 | N.A. | 15.2 | N.A. |
| 37° C., 60% | 25 | N.A. | N.A. | N.A. | 12.9 | N.A. |
| | 12.5 | N.A. | N.A. | N.A. | N.A. | N.A. |
| | 6.25 | N.A. | N.A. | N.A. | N.A. | N.A. |
| | 3.125 | N.A. | N.A. | N.A. | N.A. | N.A. |
| | 1.5626 | N.A. | N.A. | N.A. | N.A. | N.A. |
| Control | Antibiotic | 18.5 | 25.6 | N.A. | 15.2 | N.A. |
| Group | Negative | N.A. | N.A. | N.A. | N.A. | N.A. |
| | Control | | | | | |

Antibiotic: ketoconazole (100 μg/mL), tetracycline (50 μg/mL)
Negative control: distilled water (diluent)
N.A.: no activity, no influence

1-2) Antibacterial Activity of Scutellaria baicalensis Extract Fermented Under 37° C. and 50% Conditions Using S. cerevisiae (S. C.)

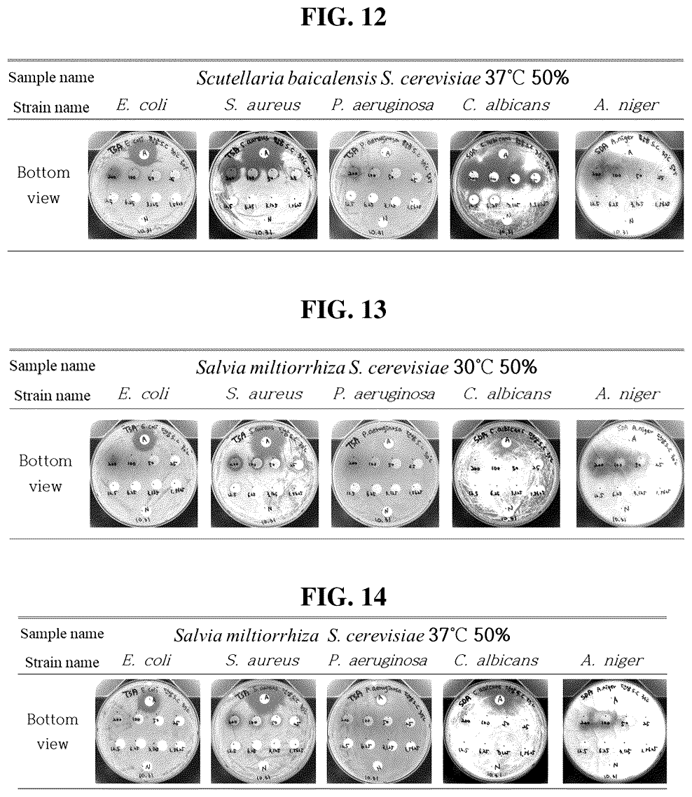

As a result of confirming the antibacterial activity of a Scutellaria baicalensis extract fermented under 37° C. and 50% conditions using S. cerevisiae (S. C.) by the disc diffusion method according to its treatment at various concentrations (1.56 mg/mL to 200 mg/mL), clear zones began to appear with a size of 8.6 mm in growth inhibition against E. coli from the concentration of 25 mg/mL and there was no distinct increase in the size of the clear zone along with the increase of the concentration, but the size of the clear zone was shown to be 12.5 mm in the group treated at the concentration of 200 mg/mL. As for the growth inhibition against C. albicans, the size of the clear zone was shown to be 9.8 mm at a low concentration of 6.25 mg/mL and the size of the clear zone became wider as the treatment concentration became higher and the clear zones were clearly shown starting from the concentration of 25 mg/mL, and the size of the clear zone was widest to be 25.1 mm at 200 mg/mL. As for the inhibition against P. aeruginosa, the size of the clear zone was shown to be 11.2 mm at 200 mg/mL. As for the inhibition against S. aureus, the clear zones began to appear starting from the concentration of 25 mg/mL, and the clear zones became wider as the concentration became higher and the size was shown to be 16.8 mm at 200 mg/mL. As for the inhibition against A. niger, the fermented Scutellaria baicalensis extract showed no effect at all regardless of its concentration (FIG. 12).

TABLE 12
Antibacterial activity of Scutellaria baicalensis extract fermented
under 37° C. and 50% conditions using S. cerevisiae (S. C.)

| | Conc. (mg) | Clear Zone (ø, mm) | | | | |
|---|---|---|---|---|---|---|
| Paper | Stock | | S. | P. | C. | A. |
| Disc | Conc. | E. coli | aureus | aeruginosa | albicans | niger |
| Scutellaria | 200 | 12.5 | 16.8 | 11.2 | 25.1 | N.A. |
| baicalensis | 100 | 10.8 | 12.3 | N.A. | 22.4 | N.A. |
| S. cerevisiae | 50 | 9.2 | 10.1 | N.A. | 18.2 | N.A. |
| (S. C.), | 25 | 8.6 | 9.4 | N.A. | 15.4 | N.A. |
| 37° C., 50% | 12.5 | N.A. | N.A. | N.A. | 10.2 | N.A. |
| | 6.25 | N.A. | N.A. | N.A. | 9.8 | N.A. |
| | 3.125 | N.A. | N.A. | N.A. | N.A. | N.A. |
| | 1.5626 | N.A. | N.A. | N.A. | N.A. | N.A. |
| Control | Antibiotic | 20.7 | 23.2 | 9.0 | 11.4 | N.A. |
| Group | Negative | N.A. | N.A. | N.A. | N.A. | N.A. |
| | Control | | | | | |

Antibiotic: ketoconazole (100 μg/mL), tetracycline (50 μg/mL)
Negative control: distilled water (diluent)
N.A.: no activity, no influence

In summary, as a result of comparing the antibacterial activity of Scutellaria baicalensis by fermenting under the conditions of 37° C. and 60% using L. casei (L. C.) and under the conditions of 37° C. and 50% using S. cerevisiae (S. C.), it was confirmed that the fermented Scutellaria baicalensis was shown to be effective against E. coli, C. albicans, P. aeruginosa, and S. aureus, and among them, the clear zones were widely distributed in C. albicans and S. aureus. However, it can be seen that the fermented Scutellaria baicalensis also has no inhibitory effect against A. niger under both conditions.

2-1). Antibacterial Activity of Salvia miltiorrhiza Extract Fermented at 30° C. Using S. cerevisiae (S. C.)

As a result of confirming the antibacterial activity of a Salvia miltiorrhiza extract fermented at 30° C. using S. cerevisiae (S. C.) by the disc diffusion method according to its treatment at various concentrations (1.56 mg/mL to 200 mg/mL), clear zones were shown to have a size of 8.6 mm at 200 mg/mL in E. coli. As for P. aeruginosa, no clear zones were shown at low concentrations but clear zones with a size of 9.6 mm appeared at 200 mg/mL. As for S. aureus, clear zones began to appear starting from 100 mg/mL, and were shown to have a size of 10.8 mm at 100 mg/mL, and 14.5 mm at 200 mg/mL, thus confirming that the clear zones became wider as the concentration increased. Additionally, no clear zones were shown in C. albicans and A. niger, thus confirming that the fermented Salvia miltiorrhiza has no inhibitory effect against the two strains (FIG. 13).

TABLE 13
Antibacterial activity of Salvia miltiorrhiza extract
fermented at 30° C. using S. cerevisiae (S. C.)

| | Conc. (mg) | Clear Zone (ø, mm) | | | | |
|---|---|---|---|---|---|---|
| Paper | Stock | | S. | P. | C. | A. |
| Disc | Conc. | E. coli | aureus | aeruginosa | albicans | niger |
| Salvia | 200 | 12.0 | 14.5 | 9.6 | N.A. | N.A. |
| miltiorrhiza | 100 | N.A. | 10.8 | N.A. | N.A. | N.A. |
| S. cerevisiae | 50 | N.A. | N.A | N.A. | N.A. | N.A. |
| (S. C.), | 25 | N.A. | N.A. | N.A. | N.A. | N.A. |
| 30° C. | 12.5 | N.A. | N.A. | N.A. | N.A. | N.A. |
| | 6.25 | N.A. | N.A. | N.A. | N.A. | N.A. |
| | 3.125 | N.A. | N.A. | N.A. | N.A. | N.A. |
| | 1.5626 | N.A. | N.A. | N.A. | N.A. | N.A. |
| Control | Antibiotic | 15.7 | 16.4 | N.A. | N.A. | N.A. |
| Group | Negative | N.A. | N.A. | N.A. | N.A. | N.A. |
| | Cont. | | | | | |

Antibiotic: ketoconazole (100 μg/mL), tetracycline (50 μg/mL)
Negative control: distilled water (diluent)
N.A.: no activitv. no influence

2-2) Antibacterial Activity of Salvia miltiorrhiza Extract Fermented at 37° C. Using S. cerevisiae (S. C.)

As a result of confirming the antibacterial activity of a Salvia miltiorrhiza extract fermented at 30° C. using S. cerevisiae (S. C.) by the disc diffusion method according to its treatment at various concentrations (1.56 mg/mL to 200 mg/mL), no clear zones were shown in E. coli, C. albicans, P. aeruginosa, and A. niger, thus confirming that the Salvia miltiorrhiza extract has no antibacterial effect against these strains. As for S. aureus, clear zones were shown to have a size of 9.4 mm at 200 mg/mL, but no effects were shown at other concentrations (FIG. 14).

TABLE 14
Antibacterial activity of Salvia miltiorrhiza extract
fermented at 37° C. using S. cerevisiae (S. C.)

| | Conc. (mg) | Clear Zone (ø, mm) | | | | |
|---|---|---|---|---|---|---|
| Paper | Stock | | S. | P. | C. | A. |
| Disc | Conc. | E. coli | aureus | aeruginosa | albicans | niger |
| Salvia | 200 | N.A | 9.4 | N.A | N.A | N.A |
| miltiorrhiza | 100 | N.A. | N.A | N.A. | N.A. | N.A. |
| S. cerevisiae | 50 | N.A. | N.A. | N.A. | N.A. | N.A. |
| (S. C.), | 25 | N.A. | N.A. | N.A. | N.A. | N.A. |
| 37° C. | 12.5 | N.A | N.A. | N.A | N.A | N.A |
| | 6.25 | N.A. | N.A | N.A. | N.A. | N.A. |
| | 3.125 | N.A. | N.A. | N.A. | N.A. | N.A. |
| | 1.5626 | N.A. | N.A. | N.A. | N.A. | N.A. |
| Control | Antibiotic | 18.5 | N.A. | N.A. | 12.4 | N.A. |
| Group | Negative | N.A. | N.A. | N.A. | N.A. | N.A. |
| | Control | | | | | |

Antibiotic: ketoconazole (100 μg/mL), tetracycline (50 μg/mL)
Negative control: distilled water (diluent)
N.A.: no activity, no influence

3-1) Antibacterial Activity of Dryopteris crassirhizoma Extract Fermented Under 30° C. and 60% Conditions Using L. casei (L. C.)

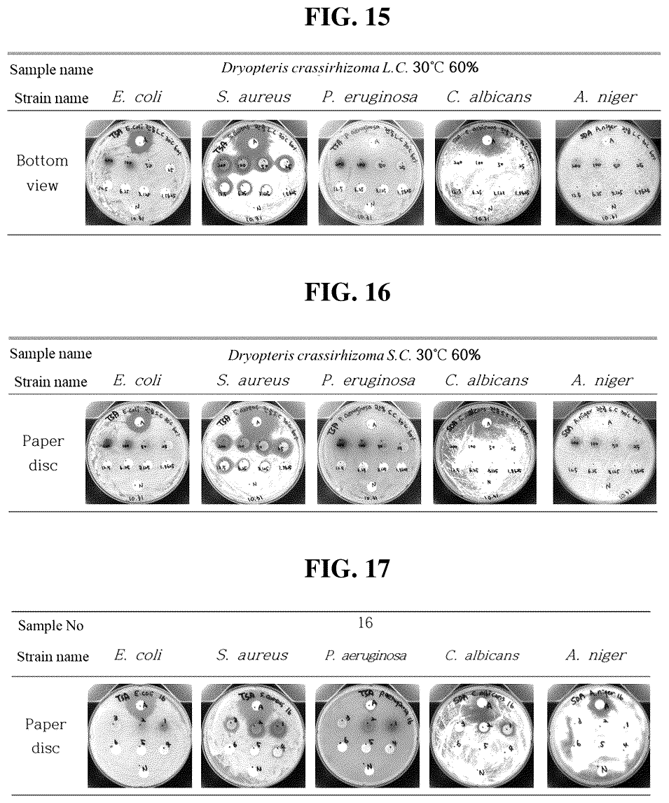

As a result of confirming the antibacterial activity of a Salvia miltiorrhiza extract fermented under 30° C. and 60% conditions using L. casei (L. C.) by the disc diffusion method according to its treatment at various concentrations (1.56 mg/mL to 200 mg/mL), clear zones in S. aureus were shown to have a size of 9.2 mm at 3.125 mg/mL, 13.3 mm at 6.25 mg/mL, 15.2 mm at 12.5 mg/mL, and 20.8 mm at 200 mg/mL, thus confirming that the Salvia miltiorrhiza extract has significant activity against S. aureus. The Salvia miltiorrhiza extract showed no antibacterial activity against E. coli, P. aeruginosa, and A. niger (FIG. 15).

TABLE 15
Antibacterial activity of Dryopteris crassirhizoma extract fermented
under 30° C. and 60% conditions using L. casei (L. C.)

| | Conc. | | | | | |
|---|---|---|---|---|---|---|
| | (mg) | Clear Zone (ø, mm) | | | | |
| Paper | Stock | | S. | P. | C. | A. |
| Disc | Conc. | E. coli | aureus | aeruginosa | albicans | niger |
| Dryopteris | 200 | N.A. | 20.8 | N.A. | N.A. | N.A. |
| crassir- | 100 | N.A | 17.2 | N.A | N.A | N.A |
| hizoma | 50 | N.A. | 15.8 | N.A. | N.A. | N.A. |
| S. cerevisiae | 25 | N.A. | 15.8 | N.A. | N.A. | N.A. |
| (S. C.), | 12.5 | N.A. | 15.2 | N.A. | N.A. | N.A. |
| 37° C., 60% | 6.25 | N.A | 13.3 | N.A | N.A | N.A |
| | 3.125 | N.A. | 9.2 | N.A. | N.A. | N.A. |
| | 1.5626 | N.A. | N.A. | N.A. | N.A. | N.A. |
| Control | Antibiotic | 17.8 | 24.2 | N.A. | N.A. | N.A. |
| Group | Negative | N.A. | N.A. | N.A. | N.A. | N.A. |
| | Control | | | | | |

Antibiotic: ketoconazole (100 μg/mL), tetracycline (50 μg/mL)
Negative control: distilled water (diluent)
N.A.: no activity, no influence

3-2). Antibacterial Activity of Dryopteris crassirhizoma Extract Fermented Under 30° C. and 60% Conditions Using S. cerevisiae (S. C.)

As a result of confirming the antibacterial activity of a Salvia miltiorrhiza extract fermented under 30° C. and 60% conditions using S. cerevisiae (S. C.) by the disc diffusion method according to its treatment at various concentrations (1.56 mg/mL to 200 mg/mL), in E. coli, clear zones appeared at 50 mg/mL and the clear zones were shown to have a size of 9.3 mm at 100 mg/mL and 9.8 mm at 200 mg/mL. In in S. aureus, the clear zones were distinctively shown to be as wide as 9.3 mm at 3.125 mg/mL, 14.2 mm at 50 mg/mL, and 15.6 mm at 200 mg/mL, thus confirming that the Salvia miltiorrhiza extract is effective against S. aureus. The Salvia miltiorrhiza extract showed no antibacterial activity against P. aeruginosa and A. niger (FIG. 16).

TABLE 16
Antibacterial activity of Dryopteris crassirhizoma extract fermented
at 30° C. and 60% conditions using S. cerevisiae (S. C.)

| | Conc. | | | | | |
|---|---|---|---|---|---|---|
| | (mg) | Clear Zone (ø, mm) | | | | |
| Paper | Stock | E. | S. | P. | C. | A. |
| Disc | Conc. | coli | aureus | aeruginosa | albicans | niger |
| Dryopteris | 200 | 9.8 | 15.6 | N.A. | N.A. | N.A. |
| crassirhizoma | 100 | 9.3 | 14.5 | N.A. | N.A. | N.A. |
| S. cerevisiae | 50 | 8.5 | 14.2 | N.A. | N.A. | N.A. |
| (S. C.), | 25 | N.A. | 11.6 | N.A. | N.A. | N.A. |
| 30° C., 60% | 12.5 | N.A. | 11.6 | N.A. | N.A. | N.A. |
| | 6.25 | N.A. | 10.8 | N.A. | N.A. | N.A. |
| | 3.125 | N.A. | 9.3 | N.A. | N.A. | N.A. |
| | 1.5626 | N.A. | N.A. | N.A. | N.A. | N.A. |
| Control | Antibiotic | 14.2 | 26.5 | 9.2 | N.A. | N.A. |
| Group | Negative | N.A. | N.A. | N.A. | N.A. | N.A. |
| | Control | | | | | |

Antibiotic: ketoconazole (100 μg/mL), tetracycline (50 μg/mL)
Negative control: distilled water (diluent)
N.A.: no activity, no influence

5. Antibacterial Activity of Fermented Complex of Scutellaria baicalensis, Salvia Miltiorrhiza, and Dryopteris crassirhizoma

After fermenting Scutellaria baicalensis, Salvia miltiorrhiza, and Dryopteris crassirhizoma according to conditions, and among the fermentation products, two from each species showing excellent antibacterial activity were selected by performing antibacterial experiments, and the antimicrobial activity was evaluated by varying the mixing ratio (Table 17). Since antibacterial activity was insignificant or not shown at all in liquid fermentation, a total of 6 types were selected from solid fermentation. The experiments were performed for each combination as such, numbers were assigned for each, and three combinations showing the most excellent efficacy among the conditions were finally selected. The resultants were prepared by measuring according to the selected conditions and dissolving in sterile water, and in the case where the resultants could not be suspended, the resultants were subjected to ultrasonic treatment at 40° C. for 20 minutes or more, filtered, and used in the experiment.

TABLE 17
Fermentation conditions and mixing ratio of antibacterial
activity according to conditions

| | Scutellaria baicalensis, | Salvia miltiorrhiza, | Dryopteris crassirhizoma, |
|---|---|---|---|
| No. | L. casei, 37° C., 60% | S. cerevisiae, 30° C., 50% | S. cerevisiae, 30° C., 60% |
| 16 | 1 | 1 | 0.5 |
| 20 | 2 | 1 | 1 |
| 24 | 1 | 2 | 1 |

### 1) Fermentation Complex-16 Evaluation of Antibacterial Activity

In the disc diffusion method of Fermentation Complex-16, the antibacterial activities of C. albicans (12 mm) and S. aureus (15 mm) were confirmed as the maximum clear zones (FIG. 17).

### 2) Fermentation Complex-20 Evaluation of Antibacterial Activity

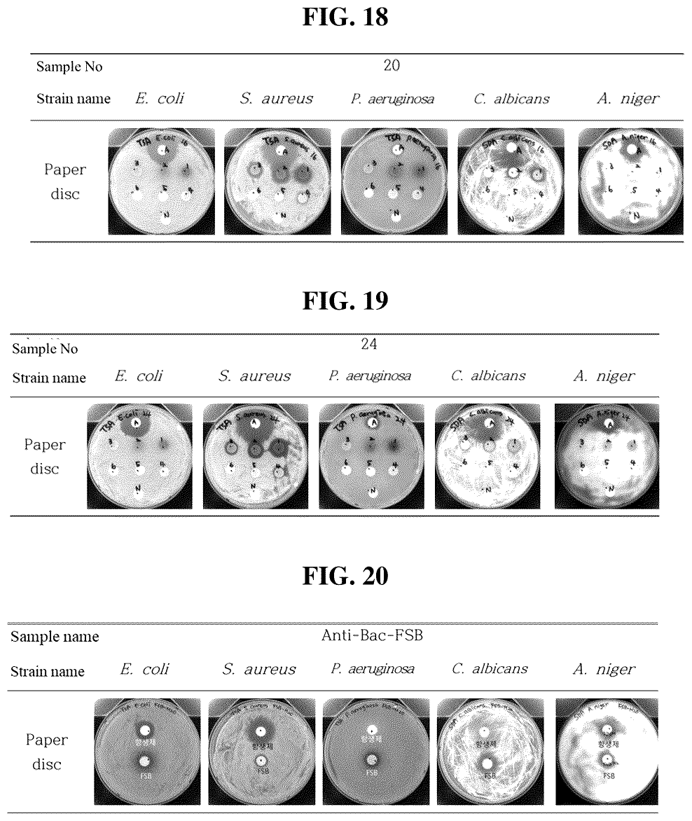

In the disc diffusion method of Fermentation Complex-20, the antibacterial activities of C. albicans (12 mm) and S. aureus (16 mm) were confirmed as the maximum clear zones (FIG. 18).

### 3) Fermentation Complex-24 Evaluation of Antibacterial Activity

In the disc diffusion method of Fermentation Complex-24, the antibacterial activities of C. albicans (10 mm) and S. aureus (17 mm) were confirmed as the maximum clear zones (FIG. 19).

### 6. Comparison of Antibacterial Activities Among Developed Natural Preservative (Anti-Bac-FSB), Commercially-Available Natural Preservatives, and Common Natural Preservatives

### 1) Commercially-Available Natural Preservatives and Common Natural Preservatives

Along with the developed natural preservative (Anti-Bac-FSB), three natural preservatives and three common preservatives were purchased and prepared according to the contents, and used in the antibacterial experiments.

TABLE 18
A list of commercially-available natural
preservatives and common preservatives

| | |
|---|---|
| Category of Preservatives | |
| (Manufacturer) | Content (%) |

| | | | | | |
|---|---|---|---|---|---|
| (common) Hexanediol | 1 | 2 | 3 | 4 | 5 |
| (common) Butylene Glycol | 5 | 10 | 15 | 20 | 25 |
| (common) Methylparaben | 0.6 | 1.3 | 2.5 | 5 | 10 |
| (natural) Company A | 5 | 10 | 15 | 20 | 25 |
| (commercially available) | | | | | |
| (natural) Company B | 5 | 10 | 15 | 20 | 25 |
| (commercially available ) | | | | | |
| (natural) Company C | 5 | 10 | 15 | 20 | 25 |
| (commercially available) | | | | | |

### 2) Evaluation of Antibacterial Activity Between Common Preservatives and Natural Preservatives

For the antibacterial experiment, the mixing ratio of Sample Condition No. 16, which was shown to have the best effect in the experiment (i.e., (Scutellaria baicalensis, L. casei, 37° C., 60%) 1: (Salvia miltiorrhiza, S. cerevisiae, 30° C., 50%) 1: (Dryopteris crassirhizoma, S. cerevisiae, 30° C., 60%) 0.5), was selected as the product name was assigned as Anti-Bac-FSB. Anti-Bac-FSB, commercially-available antibiotics with a preservative effect, commercially-available natural preservatives, and common preservatives were injected into paper discs to perform the antibacterial experiment.

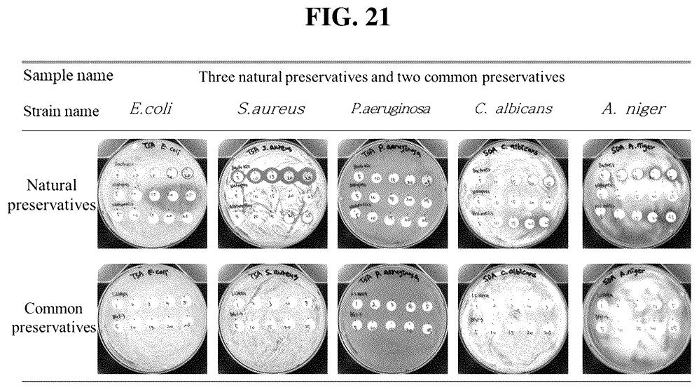

A comparison experiment was performed using the product which was developed in this study (product name: Anti-Bac-FSB) in which each of a fermented extract of Scutellaria baicalensis, a fermented extract of Salvia miltiorrhiza, and a fermented extract of Dryopteris crassirhizoma having the best antibacterial effect were mixed, three commercially-available preservatives, two common preservatives, and as an antibiotic tetracycline was used for three bacterial strains (Escherichia coli, Staphylococcus aureus, and Pseudomonas aeruginosa) and ketoconazole was used for the two fungal strains (Candida albicans and Aspergillus niger), and the results are as follows (FIGS. 20 and 21).

In the clear zone experiment with respect to the three bacterial strains (E. coli, S. aureus, and P. aeruginosa), Anti-Bac-FSB and the antibiotic showed excellent antibacterial activity against E. coli by showing the size of 12.8 mm and 14.2 mm, respectively, whereas the product A, which is commercially-available, showed a clear zone of 10.0 mm thus showing a somewhat low result, and the remaining products from Company B and Company C and common preservatives showed no antibacterial effect. As for S. aureus, the antibiotic showed the highest size of 18.2 mm, Anti-Bac-FSB and the product from Company A showed high sizes of 11.4 mm and 13.8 mm, the product from Company C showed a low size of 9.3 mm, and the product from Company B and common preservatives showed no antibacterial activity. As for P. aeruginosa, Anti-Bac-FSB showed wide clear zones with a size of 13.2 mm, but the antibiotic, commercially-available preservatives, and common preservatives showed no antibacterial activity.

In the experiment with respect to two fungal strains (C. albicans and A. niger), Anti-Bac-FSB showed the widest size of 15.7 mm in C. albicans, thus confirming that Anti-Bac-FSB has excellent antibacterial activity, whereas the product from Company A showed a smaller size of 10.0 mm and the antibiotic and other different preservatives showed no antibacterial activity. In the experiment with respect to A. niger, Anti-Bac-FSB showed the widest size of 14.8 mm followed by 13.2 mm by the antibiotic, whereas the product from Company A showed antibacterial activity but the clear zone was somewhat narrow with a size of 10.1 mm, and the products from Company B and Company C and the two common preservatives showed no antibacterial activity.

TABLE 19
Comparison of antibacterial activities among test natural preservative
(Anti-Bac-FSB), commercially-available natural preservatives,
and common natural preservatives

| | | Strain | | | | |
|---|---|---|---|---|---|---|
| | | Clear zone (ø, mm) | | | | |
| | | E. | S. | P. | C. | A. |
| Items | | coli | aureus | aeruginosa | albicans | niger |
| Paper | Anti-Bac-FSB | 12.8 | 11.4 | 13.2 | 15.7 | 14.8 |
| Disc | Antibiotic | 14.2 | 18.2 | N.A. | N.A. | 13.2 |
| | Company A | 10.0 | 13.8 | N.A. | 10.0 | 10.1 |
| | Company B | N.A | N.A. | N.A | N.A | N.A |
| | Company C | N.A. | 9.3 | N.A. | N.A. | N.A. |
| | 1.2 hexanediol | N.A. | N.A. | N.A. | N.A. | N.A. |
| | 1-3 BG | N.A. | N.A. | N.A. | N.A. | N.A. |

Anti-Bac-FSB: natural preservative developed by the applicant's company
Antibiotic: ketoconazole (200 μg/mL), tetracycline (100 μg/mL)
μg/mL

Through the results of Experimental Examples and Examples above, it was confirmed that while Anti-Bac-FSB of the present invention showed an inhibitory effect against bacteria and fungi, the product of Company A showed a low level of antibacterial activity in strains other than P. aeruginosa, and other strains showed no antibacterial activity at all. In the case of fungal strains, it was confirmed that while a single use of Scutellaria baicalensis, Salvia miltiorrhiza, and Dryopteris crassirhizoma showed no antibacterial activity at all, a mixture thereof increased the antibacterial activity.

Accordingly, it was confirmed that as for the natural preservatives, when Scutellaria baicalensis, Salvia miltiorrhiza, and Dryopteris crassirhizoma were mixed after solid fermentation, they showed remarkable excellent antibacterial activity compared to commercially-available natural preservatives.

The foregoing description of the present invention is for illustrative purposes only, and those skilled in the art to which the present invention pertains will understand that it can easily be modified into other specific forms without changing the technical spirit or essential characteristics of the present invention. Therefore, the embodiments described above should be understood as illustrative in all respects and not limiting. For example, each component described as a single type may be implemented in a distributed manner, and similarly, components described as distributed may be implemented in a combined form.

The scope of the present invention is indicated by the following claims, and all changes or modifications derived from the meaning and scope of the claims and equivalent concepts thereof should be construed as being included in the scope of the present invention.

1. A composition having antibacterial or antifungal activity, which comprises a fermented extract of Scutellaria baicalensis, a fermented extract of Salvia miltiorrhiza, and a fermented extract of Dryopteris crassirhizoma.

2. The composition of claim 1, wherein the fermented extracts are each fermented by solid fermentation.

3. The composition of claim 1, wherein the fermented extracts are each fermented by any one of bacteria of the genus Lactobacillus and fungi of the genus Saccharomyces.

4. The composition of claim 1, wherein the fermented extracts are each fermented by any one of Lactobacillus casei (L. casei) and Saccharomyces cerevisiae (S. cerevisiae).

5. The composition of claim 1, wherein the fermented extracts are each fermented at a temperature between 20° C. and 40° C.

6. The composition of claim 1, wherein the fermented extracts are each fermented under the condition where the amount of water added is between 40% to 70%.

7. The composition of claim 1, wherein the weight mixing ratio between the fermented extract Scutellaria baicalensis: the fermented extract of Salvia miltiorrhiza: the fermented extract of Dryopteris crassirhizoma is 1:0.5 to 2:0.5 to 1.

8. The composition of claim 1, wherein the composition has antibacterial activity against Escherichia coli, Staphylococcus aureus, or Pseudomonas aeruginosa.

9. The composition of claim 1, wherein the composition has antifungal activity against Candida albicans or Aspergillus niger.

10. The composition of claim 1, wherein the composition is used in one or more selected from the group consisting of cosmetics, skin cleaners, hair cleaners, vaginal cleaners, bathroom cleaners, mouthwashes, kitchen cleaners, cosmetic preservatives, food preservatives, pharmaceutical preservatives, food additives, and feed additives.

11. A method for preparing a composition having antibacterial or antifungal activity, which comprises:
fermenting each of Scutellaria baicalensis, Salvia miltiorrhiza, and Dryopteris crassirhizoma by Lactobacillus casei (L. casei) and Saccharomyces cerevisiae (S. cerevisiae) and obtaining fermented extracts thereof;
mixing the fermented extracts, diluting the mixture in sterile water, and treating the resultant with ultrasonic waves at 40° C. for 20 to 30 minutes; and
filtering the mixed fermented extract.

12. The method of claim 11, wherein each of the Scutellaria baicalensis, Salvia miltiorrhiza, and Dryopteris crassirhizoma is fermented in a solid state.

13. The method of claim 11, wherein in the composition, the weight mixing ratio between the fermented extract Scutellaria baicalensis: the fermented extract of Salvia miltiorrhiza: the fermented extract of Dryopteris crassirhizoma is 1:0.5 to 2:0.5 to 1.

14. The method of claim 11, wherein the fermented extracts are each fermented by solid fermentation under the condition where the amount of water added at a temperature between 20° C. and 40° C. is 40% to 70%.
